SEQUENCE LISTING (16 sequences)

SEQ ID NO: 1 - protein, 446 aa, Artificial Sequence
  Synthetic
QVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, Artificial Sequence
  Synthetic
DIQMTQSPSSLSASVGDRVTITCRASEDVNTYVSWYQQKPGKAPKLLIYAASNRYTGVPSRFSGSGSGTDFTLTISSLQPEDFATYYCQQSFSYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 449 aa, Artificial Sequence
  Synthetic
QVQLVQSGAEVKKPGSSVKVSCKASGYTFTDNYMIWVRQAPGQGLEWMGDINPYNGGTTFNQKFKGRVTITADKSTSTAYMELSSLRSEDTAVYYCARESPYFSNLYVMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 218 aa, Artificial Sequence
  Synthetic
EIVLTQSPATLSLSPGERATLSCKASQSVDYDGDNYMNWYQQKPGQAPRLLIYAASNLESGIPARFSGSGSGTDFTLTISSLEPEDFAVYYCHLSNEDLSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 450 aa, Artificial Sequence
  Synthetic
QDQLVESGGGVVQPGRSLRLSCAASGFTFSSYGMHWVRQAPGEGLEWVAVIWYDGSNKYYADSVKGRFTISRDNSKNTLYLQMNSLRAEDTAVYYCARGDYEVDYGMDVWGQGTTVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, Artificial Sequence
  Synthetic
AIQLTQSPSSLSASVGDRVTITCRASQGISNALAWYQQKPGKAPKLLIYDASSLESGVPSRFSGSGSGTDFTLTISSLQPEDFATYYCQQFNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 446 aa, Artificial Sequence
  Synthetic
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARESWFGEVFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 8 - protein, 220 aa, Artificial Sequence
  Synthetic
DIVMTQSPDSLAVSLGERATINCKSSQSVLDSSDNKNYLAWYQQKPGQPPKLLIYWASNRESGVPDRFSGSGSGTDFTLTISSLQAEDVAVYYCQQYYSDPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 453 aa, Artificial Sequence
  Synthetic
EVTLKESGPALVKPTQTLTLTCTFSGFSLTTYGMGVGWIRQPPGKALEWLANIWWDDDKYYNPSLKNRLTISKDTSKNQVVLTMTNMDPVDTATYYCARIGPIKYPTAPYRYFDFWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, Artificial Sequence
  Synthetic
DIQMTQSPSSLSASVGDRVTITCLASEDIYDNLAWYQQKPGKAPKLLIYYASSLQDGVPSRFSGSGSGTDYTLTISSLQPEDFATYYCLQDSEYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 448 aa, Artificial Sequence
  Synthetic
QVQLQESGPGLVKPSQTLSLTCTVSDYSITSDYAWNWIRQFPGKGLEWMGYISYSGSTSYNPSLKSRITISRDTSKNQFSLQLNSVTAADTAVYYCASFDYAHAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 214 aa, Artificial Sequence
  Synthetic
DIVLTQSPAFLSVTPGEKVTFTCQASQSIGTSIHWYQQKTDQAPKLLIKYASESISGIPSRFSGSGSGTDFTLTISSVEAEDAADYYCQQINSWPTTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 447 aa, Artificial Sequence
  Synthetic
DVQLQESGPGLVKPSQSLSLTCTVTDYSITSDYAWNWIRQFPGNKLEWMGYISYSGSTSYNPSLKSRISITRDTSKNQFFLQLNSVTTEDTATYYCASFDYAHAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 14 - protein, 214 aa, Artificial Sequence
  Synthetic
DILLTQSPAILSVSPGERVSFSCRASQSIGTSIHWYQQRTNGSPRLLIKYASESISGIPSRFSGSGSGTDFTLSINSVESEDIADYYCQQINSWPTTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 15 - protein, 447 aa, Artificial Sequence
  Synthetic
EVQLVESGGGLVQPGGSLRLSCAASGFTFSSFSMTWVRQAPGKGLEWVSYISSRSSTISYADSVKGRFTISRDNAKNSLYLQMNSLRDEDTAVYYCARDPLLAGATFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 215 aa, Artificial Sequence
  Synthetic
EFVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0199752 A1
(43) Pub. Date: Jun. 20, 2024

(54) TREATMENT OF OPHTHALMIC DISEASES

(71) Applicants: Kirk William JOHNSON, Redwood City, CA (US); Laman ALANI, Redwood City, CA (US); AmMax Bio, Inc., Redwood City, CA (US)

(72) Inventors: Kirk William JOHNSON, Moraga, CA (US); Laman ALANI, Menlo Park, CA (US)

(21) Appl. No.: 18/556,627

(22) PCT Filed: Apr. 22, 2022
(86) PCT No.: PCT/US2022/026006

§ 371 Date: Oct. 20, 2023

Related U.S. Application Data
(60) Provisional application No. 63/178,996, filed on Apr. 23, 2021.
(60) Provisional application No. 63/311,214, filed on Feb. 17, 2022.

(51) Int. Cl. C07K 16/28; A61K 39/00; A61P 27/02; C07K 16/22

(52) U.S. Cl. CPC C07K 16/2866; A61P 27/02; C07K 16/22; A61K 2039/505; C07K 2317/31

(57) ABSTRACT

Provided herein are pharmaceutical compositions and related methods for treating ophthalmic diseases. The method entails administering to the patient an effective amount of a CSF1R inhibitor, such as an anti-CSF1R antibody. The methods are effective in reducing vascular hyperpermeability, neovascularization and fibrosis such as macular fibrosis. Combination therapies that also include a VEGF inhibitor are also provided. Preferably, the administration is via intravitreal injection.

### CROSS REFERENCE TO RELATED APPLICATIONS

This application claims the benefit under 35 U.S.C. § 119(e) of the U.S. Provisional Application Ser. No. 63/178,996 filed Apr. 23, 2021 and 63/311,214 filed Feb. 17, 2022, the content of each of which is hereby incorporated by reference in its entirety.

### SEQUENCE LISTING

The instant application contains a Sequence Listing which has been submitted in ASCII format via EFS-Web and is hereby incorporated by reference in its entirety. Said ASCII copy, created on Apr. 22, 2022, is named 329305_SL.txt and is 5,775 bytes in size.

### BACKGROUND

Choroidal neovascularization (CNV) is new abnormal blood vessels emerging from the choroid and growing through the Bruch membrane and sometimes the retinal pigment epithelium (RPE). CNV is observed during neovascular age-related macular degeneration (nAMD) and can lead to vision loss in patients experiencing AMD.

In the wet, or exudative, form of age-related macular degeneration (AMD or ARMD), pathologic choroidal neovascular membranes (CNVM) develop under the retina. The CNVM can leak fluid and blood and, if left untreated, ultimately cause a centrally blinding disciform scar. Approximately 10-20% of patients with nonexudative AMD eventually progress to the exudative form, which is responsible for the majority of the estimated 1.75 million cases of advanced AMD in the United States.

Diabetic macular edema (DME) is caused by a complication of diabetes called diabetic retinopathy. Diabetic retinopathy, including proliferative diabetic retinopathy (PDR) is the most common diabetic eye disease and the leading cause of irreversible blindness in working age Americans. Diabetic retinopathy usually affects both eyes. Diabetic retinopathy is caused by ongoing damage to the small blood vessels of the retina. The leakage of fluid into the retina may lead to swelling of the surrounding tissue, including the macula.

DME is the most common cause of vision loss in people with diabetic retinopathy. Poor blood sugar control and additional medical conditions, such as high blood pressure, increase the risk of blindness for people with DME. DME can occur at any stage of diabetic retinopathy, although it is more likely to occur later as the disease goes on. Experts estimate that approximately 7.7 million Americans have diabetic retinopathy and of those, about 750,000 also have DME.

New and improved treatments of these diseases and conditions, and more generally ophthalmic diseases, are needed.

### SUMMARY

Accordingly, one embodiment of the disclosure provides a method for treating an ophthalmic disease in a human patient in need thereof. The method may entail administering to the patient an effective amount of an anti-CSF1R agent such as an inhibitory anti-CSF1R antibody.

In some embodiments, the ophthalmic disease is characterized with vascular hyperpermeability. In some embodiments, the ophthalmic disease is characterized with neovascularization. The ophthalmic disease may also involve fibrosis such as macular fibrosis.

Non-limiting examples of ophthalmic diseases include age-related macular degeneration (AMD), anterior segment neovascularization, central retinal vein occlusion (CRVO), choroidal neovascularization (CNV), corneal neovascularization, diabetic macular edema (DME), diabetic retinopathy, dry eye syndrome, glaucoma, noninfectious uveitis (NIU), polypoidal choroidal vasculopathy, posterior segment neovascularization, proliferative diabetic retinopathy (PDR), proliferative vitreoretinopathy, retinal edema, retinal vein occlusion (RVO), retinopathy of prematurity (ROP), sequela associated with retinal ischemia, and uveitis.

In some embodiments, the method further comprises administering a VEGF inhibitor, such as an antibody. In some embodiments, the method may entail a combination therapeutic of a VEGF inhibitor and an anti-CSF1R antibody. In some embodiments, the administration is via intravitreal injection, which can be once every one, two, three, four, five, or six months.

These and other embodiments are further described in the text that follows.

### BRIEF DESCRIPTION OF THE DRAWINGS

FIG. 1 illustrates the experimental procedure of using a laser-induced choroidal neovascularization (LCNV) mouse model to test the effectiveness of wetAMD treatments.

FIG. 2 shows the impacts of the different treatments in reducing choroidal neovascularization area.

FIG. 3 shows the impacts of the different treatments in reducing vascular permeability in the LCNV model.

### DETAILED DESCRIPTION

### Definitions

Unless defined otherwise, all technical and scientific terms used herein have the same meaning as is commonly understood by one of ordinary skill in the art. As used herein, the below terms have the following meanings unless specified otherwise. Any methods, devices and materials similar or equivalent to those described herein can be used in the practice of the compositions and methods described herein. The following definitions are provided to facilitate understanding of certain terms used frequently herein and are not meant to limit the scope of the present disclosure. All references referred to herein are incorporated by reference in their entirety.

Headings used in this application are for reference purposes only and do not in any way limit the present disclosure.

As used herein, the term “comprising” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this invention.

The term “% w/w” refers to the percent of the weight of a component based on the total weight of a composition comprising the component unless explicitly stated otherwise. For instance, if component 1 is present in an amount of 50 mg in a 100 mg composition, component 1 is present in an amount of 50% w/w. Percent weights described herein do not include the weight of a container unless explicitly stated as such.

The term “administration” refers to introducing an agent into a patient. An effective amount can be administered, which can be determined by the treating physician or the like. The related terms and phrases administering” and “administration of”, when used in connection with a compound or tablet (and grammatical equivalents) refer both to direct administration, which may be administration to a patient by a medical professional or by self-administration by the patient.

“Therapeutically effective amount” or “effective amount” refers to an amount of a drug or an agent that when administered locally via a pharmaceutical composition described herein to a patient suffering from a condition, will have an intended therapeutic effect, e.g., alleviation, amelioration, palliation or elimination of one or more symptoms of the condition in the patient. The full therapeutic effect does not necessarily occur immediately and may occur only after a therapeutically effective amount is being delivered continuously for a period of time. For slow release or controlled release formulation, “therapeutically effective amount” or “effective amount” may refer to the total amount that is effective over a period of time, which is slowly released from the delivery vehicle to the disease site at an ascertainable and controllable release rate that constantly provides an effective amount of the drug to the disease site. In some embodiments, “therapeutically effective amount” or “effective amount” refers to an amount released to the disease site at a given period of time, e.g., per day.

The term “pharmaceutically acceptable” refers to generally safe and non-toxic for human administration.

“Treatment”, “treating”, and “treat” are defined as acting upon a disease, disorder, or condition with an agent to reduce or ameliorate the harmful or any other undesired effects of the disease, disorder, or condition and/or its symptoms.

Unless otherwise specified, the terms “drug,” “active ingredient,” “active pharmaceutical ingredient,” “therapeutic agent” and “API” are used synonymously to refer to the component in the composition that has a desired therapeutic effect.

“Antibody” means a human or non-human antibody, including humanized antibodies, and may be polyclonal or monoclonal, and/or chimeric antibodies. The term “antibody” includes antibody fragments capable of binding to antigen and may be selected from Fab, an Fv, an scFv, Fab′ and Fab″. The antibody may be of any isotype. The antibody can be wild-type or can include one or more mutations. For example, the mutation may be a conservative substitution of a cysteine residue. An “anti-CSF1R antibody” has the corresponding meaning with respect to an antibody to the CSF1R receptor.

Colony stimulating factor 1 (CSF-1, CSF1), also known as macrophage colony stimulating factor (M-CSF), is a cytokine produced by a variety of cells, including macrophages, endothelial cells and fibroblasts. CSF-1 is composed of two “monomer” polypeptides, which form a biologically active dimeric CSF-1 protein. CSF-1 exists in at least three mature forms due to alternative RNA splicing (see, Cerretti et al. Molecular Immunology, 25:761 (1988)). The three forms of CSF-1 are translated from different rnRNA precursors, which encode polypeptide monomers of 256 to 554 amino acids, having a 32 amino acid signal sequence at the amino terminal and a putative transmembrane region of approximately 23 amino acids near the carboxyl terminal. The precursor peptides are subsequently processed by amino terminal and carboxyl terminal proteolytic cleavages to release mature CSF-1. Residues 1-149 of all three mature forms of CSF-1 are identical and are believed to contain sequences essential for biological activity of CSF-1. CSF-1 monomers are dimerized in vivo via disulfide-linkage and are glycosylated. CSF-1 belongs to a group of biological agonists that promote the production of blood cells. Specifically, it acts as a growth and differentiation factor for bone marrow progenitor cells of the mononuclear phagocyte lineage.

Colony stimulating factor 1 receptor (referred to herein as CSF1R; also referred to as FMS, FIM2, C-FMS, or CD115) is a single-pass transmembrane receptor with an N-terminal extracellular domain (ECD) and a C-terminal intracellular domain with tyrosine kinase activity. CSF1R belongs to the type III protein tyrosine kinase receptor family, and binding of CSF1 or the interleukin 34 ligand induces homodimerization of the receptor and subsequent activation of receptor signaling. CSF1R-mediated signaling is crucial for the differentiation and survival of the mononuclear phagocyte system and macrophages in particular.

“CSF1R inhibitor” refers to a compound, that inhibits the activity of a colony-stimulating factor 1 receptor (CSF1R). The compound can be either a small molecule, such as pexidartinib, imatinib, quizartinib, sunitinib, lestaurtinib, midostaurin, tandutinib, sorafenib, ponatinib, ARRY-382, AC708, JNJ-40346527, BLZ945, CYC10268, AZ683, OSI-930, DCC-2618, DCC-3014, PLX7486, ABT-869, AG013736, Ki20227, GW2580, those described in U.S. Pat. No. 5,710,158, and US2017/0157118, or a large molecule, for example an antibody, such as those provided in Table 1A. Also included are nucleic acid inhibitors, such as siRNA or antisense polynucleotides.

“CSF1 inhibitor” refers to a compound, that inhibits the activity of a colony-stimulating factor 1 (CSF1). The compound can be either a small molecule or a large molecule, for example an antibody. Examples include PD-0360324 and MCS110, and anti-CSF1 antibodies, such as those provided in Table 1B. Also included are nucleic acid inhibitors, such as siRNA or antisense polynucleotides.

“Controlled release”, “sustained release”, or “slow release” and similar terms are used to denote a mode of active agent delivery that occurs when the active agent is released from the delivery vehicle over a period of time, rather than being dispersed immediately (e.g., at a diffusion-controlled rate) upon application or injection.

### Treatment of Ophthalmic Diseases

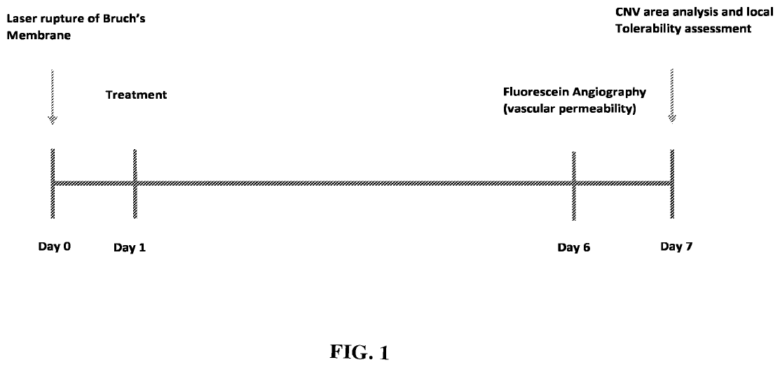

The present disclosure presents experimental data demonstrating the efficacy of anti-CSF1R antibodies in treating wet, or exudative, age-related macular degeneration (wetAMD) in a LCNV (laser-induced choroidal neovascularization) mouse model. As shown in FIG. 1, like the reference anti-VEGF antibody, the anti-CSF1R antibody was also able to significantly attenuate choroidal neovascularization in a dose-dependent manner and with maximal efficacy at least comparable to a potent anti-VEGF antibody reference control.

Quite unexpectedly, however, the anti-CSF1R antibody was significantly more effective than the anti-VEGF antibody in reducing vascular permeability. Such a finding is uncommon in this type of model amongst those familiar with this disease model and skilled in the art. Vascular hyperpermeability is characteristic of certain ophthalmic diseases and certain stages/patients of ophthalmic diseases. Therefore, the data support the use of anti-CSF1R antibodies in treating such diseases.

The disclosure also provides examples testing the efficacy of anti-CSF1R antibodies in other ophthalmic diseases such as proliferative diabetic retinopathy (PDR), and diabetic macular edema (DME). It is contemplated that the anti-CSF1R antibodies are effective in treating these diseases as well.

Moreover, in preliminary studies, it has been observed that anti-CSF1R antibodies are able to prevent and reduce fibrosis in the eye. Fibrosis is present in certain ophthalmic diseases, and in certain stages of the ophthalmic diseases, that has been shown to an obstacle to the effectiveness of certain other medications (e.g., anti-VEGF therapies). The ability of anti-CSF1R antibodies to prevent/reduce fibrosis in the eye make them promising new alternative therapies, or supplemental to existing therapies.

It is further contemplated that the combination of an anti-CSF1R antibody and an anti-VEGF therapy can further improve the effectiveness of the treatment of ophthalmic diseases. Also contemplated is that the anti-CSF1R antibody, once intravitreally injected, has a relatively long half-life and extended pharmacologic activity in the eye.

In accordance with one embodiment of the present disclosure, provided is a method of treating an ophthalmic disease in a human patient in need thereof. The method may entail administering to the patient an effective amount of a CSF1R inhibitor or a CSF1 inhibitor, such as antibodies.

In some embodiments, the ophthalmic disease is characterized with vascular hyperpermeability. In some embodiments, the ophthalmic disease is characterized with neovascularization.

Non-limiting examples of ophthalmic diseases include age-related macular degeneration (AMD), anterior segment neovascularization, central retinal vein occlusion (CRVO), choroidal neovascularization (CNV), corneal neovascularization, diabetic macular edema (DME), diabetic retinopathy, dry eye syndrome, glaucoma, noninfectious uveitis (NIU), polypoidal choroidal vasculopathy, posterior segment neovascularization, proliferative diabetic retinopathy (PDR), proliferative vitreoretinopathy, retinal edema, retinal vein occlusion (RVO), retinopathy of prematurity (ROP), sequela associated with retinal ischemia, and uveitis.

### AMD

“Macular degeneration,” also known as “age-related macular degeneration” (AMD or ARMD), is a medical condition which may result in blurred or no vision in the center of the visual field. Macular degeneration typically occurs in older people. Genetic factors and smoking also play a role. It is due to damage to the macula of the retina. Diagnosis is by a complete eye exam. The severity is divided into early, intermediate, and late types. The late type is additionally divided into “dry” and “wet” forms with the dry form making up 90% of cases.

There is no cure or treatment that returns vision already lost. In the wet form, anti-VEGF medication injected into the eye or less commonly laser coagulation or photodynamic therapy may slow worsening. Dietary supplements may also slow the progression in those who already have the disease.

AMD can be divided into 3 stages: early, intermediate, and late, based partially on the extent (size and number) of drusen, which is the characteristic yellow deposits.

In some embodiments, the AMD is early AMD, which is diagnosed based on the presence of medium-sized drusen, about the width of an average human hair. Early AMD is usually asymptomatic.

In some embodiments, the AMD is intermediate AMD, which is diagnosed by large drusen and/or any retinal pigment abnormalities. Intermediate AMD may cause some vision loss, but, like early AMD, it is usually asymptomatic.

In some embodiments, the AMD is late AMD. In late AMD, enough retinal damage occurs that, in addition to drusen, people will also begin to experience symptomatic central vision loss. The damage can either be the development of atrophy or the onset of neovascular disease. Late AMD is further divided into two subtypes based on the types of damage: Geographic atrophy and Wet AMD (also called Neovascular AMD).

In some embodiments, the AMD is geographic atrophy (also called atrophic AMD). Geographic atrophy is an advanced form of AMD in which progressive and irreversible loss of retinal cells leads to a loss of visual function. There are multiple layers that make up the retina, and in geographic atrophy, there are three specific layers that undergo atrophy: the choriocapillaris, retinal pigment epithelium, and the overlying photoreceptors.

In some embodiments, the AMD is wet AMD (or wetAMD). wetAMD causes vision loss due to abnormal blood vessel growth (choroidal neovascularization) in the choriocapillaris, through Bruch's membrane. It is usually, but not always, preceded by the dry form of AMD. The proliferation of abnormal blood vessels in the retina is stimulated by vascular endothelial growth factor (VEGF). Because these blood vessels are abnormal, these are also more fragile than typical blood vessels, which ultimately leads to blood and protein leakage below the macula. Bleeding, leaking, and scarring from these blood vessels eventually cause irreversible damage to the photoreceptors and rapid vision loss if left untreated.

### Corneal Neovascularization, Anterior and Posterior Segment Neovascularization

“Corneal neovascularization” is the in-growth of new blood vessels from the pericorneal plexus into avascular corneal tissue as a result of oxygen deprivation. Maintaining avascularity of the corneal stroma is an important aspect of corneal pathophysiology as it is required for corneal transparency and optimal vision. A decrease in corneal transparency causes visual acuity deterioration. Corneal tissue is avascular in nature and the presence of vascularization, which can be deep or superficial, is always pathologically related.

CNV causes may be congenital in nature, such as with Aniridia, or acquired. Frequently, inflammatory, infectious, degenerative, traumatic or iatrogenic (e.g. contact lenses) conditions can be responsible for acquired CNV. Some major acquired inflammatory conditions include graft rejection following keratoplasty, graft or host diseases of the new tissue, atopic conjunctivitis, rosacea, ocular pemphigoid, Lyell's syndrome, and Steven's Johnson syndrome.

Infections responsible for CNV range from bacterial (chlamydia, syphilis, pseudomonas), viral (herpes simplex & herpes zoster viruses), fungal (candida, aspergillus, fusarium), to parasitic (onchocerca volvolus) infection. Degenerative diseases such as pterygiums and terrien's marginal degeneration may also be responsible. Traumatic causes of CNV include ulceration, alkali burns, and stem cell deficiency.

One of the most common causes of corneal neovascularization is iatrogenic pathology from extended contact lens wear. This is especially likely with lenses made with older hydrogel materials such as HEMA (2-hydroxyethyl methacrylate) for both daily and extended wear. Such older hydrogel materials have a relatively low oxygen transmissibility so the cornea becomes starved of oxygen; this leads to the ingress of blood capillaries into the clear cornea, in an attempt to provide more oxygen to the affected area. Older estimates cite 128,000 to 470,000 cases of lens-induced CNV each year, but this may be decreasing due to the increasing popularity of daily disposable lenses.

The risk for CNV is elevated in certain instances for patients following penetrating keratoplasty without active inflammation or epithelial defects. For example, the condition is more likely to occur in those with active blepharitis, those who receive sutured knots in their host stromas, and those with a large recipient area.

Anterior segment neovascularization, like posterior segment neovascularization, is a consequence of retinal ischemia mediated by increased intraocular levels of vascular endothelial growth factor (VEGF). It differs in that it generally requires higher levels of VEGF to induce anterior segment neovascularization than to induce posterior segment neovascularization. Because levels of VEGF correlate with area of retinal ischemia, it is rare for anterior segment neovascularization (ASNV) to arise after branch retinal vein occlusion (BRVO).

### Central Retinal Vein Occlusion

“Central retinal vein occlusion” (CRVO) is when the central retinal vein becomes occluded, usually through thrombosis. The central retinal vein is the venous equivalent of the central retinal artery and both may become occluded. Since the central retinal artery and vein are the sole source of blood supply and drainage for the retina, such occlusion can lead to severe damage to the retina and blindness, due to ischemia (restriction in blood supply) and edema (swelling). CRVO can cause ocular ischemic syndrome. Nonischemic CRVO is the milder form of the disease. It may progress to the more severe ischemic type. CRVO can also cause glaucoma.

### Retinal Edema and Diabetic Macular Edema (DME)

“Retinal edema” occurs when fluid and protein deposits collect on or under the macula of the eye (a yellow central area of the retina) and causes it to thicken and swell (edema). The swelling may distort a person's central vision, because the macula holds tightly packed cones that provide sharp, clear, central vision to enable a person to see detail, form, and color that is directly in the center of the field of view.

The causes of retinal edema are numerous and different causes may be inter-related. It is commonly associated with diabetes. Chronic or uncontrolled diabetes type 2 can affect peripheral blood vessels including those of the retina which may leak fluid, blood and occasionally fats into the retina causing it to swell.

Age-related macular degeneration may cause macular edema. As individuals age there may be a natural deterioration in the macula which can lead to the depositing of drusen under the retina sometimes with the formation of abnormal blood vessels.

Replacement of the lens as treatment for cataract can cause pseudophakic macular edema. (‘pseudophakia’ means ‘replacement lens’) also known as Irvine-Gass syndrome The surgery involved sometimes irritates the retina (and other parts of the eye) causing the capillaries in the retina to dilate and leak fluid into the retina. Less common today with modern lens replacement techniques.

Blockage of a vein in the retina can cause engorgement of the other retinal veins causing them to leak fluid under or into the retina. The blockage may be caused, among other things, by atherosclerosis, high blood pressure and glaucoma.

“Diabetic macular edema” (DME) is a complication of diabetes. People with type 1 or type 2 diabetes can develop DME. DME occurs when excess fluid starts to build up in the macula of the eye. It's located in the center of the retina, the lining at the back of the eye that's full of blood vessels. When excess fluid builds up in the macula, it causes vision problems.

The exact pathogenesis of DME is still unclear. Recent evidence indicates that diabetic retinopathy (DR) is a neurovascular disease of the retina. Retinal neuronal abnormalities are present well before the retinal microvascular injury. Increased vasopermeability occurs as a result of breakdown of the BRB due to many factors: altered glial cells, loss of pericytes, endothelial cell death, leukostasis in the retinal vasculature, poor function of the tight junctions in the retinal vasculature, activation of the AGE receptor, upregulation of the expression of vascular endothelial growth factor (VEGF) and protein kinase C (PKC), and altered vitreo-retinal interface with a thickened taut, posterior hyaloid with persistent vitreo-macular traction (VMT).

### Diabetic Retinopathy and Proliferative Diabetic Retinopathy (PDR)

“Diabetic retinopathy,” also known as diabetic eye disease (DED), is a medical condition in which damage occurs to the retina due to diabetes mellitus. Diabetic retinopathy is the result of damage to the small blood vessels and neurons of the retina. The earliest changes leading to diabetic retinopathy include narrowing of the retinal arteries associated with reduced retinal blood flow; dysfunction of the neurons of the inner retina, followed in later stages by changes in the function of the outer retina, associated with subtle changes in visual function; dysfunction of the blood-retinal barrier, which protects the retina from many substances in the blood (including toxins and immune cells), leading to the leaking of blood constituents into the retinal neuropile. Later, the basement membrane of the retinal blood vessels thickens, capillaries degenerate and lose cells, particularly pericytes and vascular smooth muscle cells. This leads to loss of blood flow and progressive ischemia, and microscopic aneurysms which appear as balloon-like structures jutting out from the capillary walls, which recruit inflammatory cells; and advanced dysfunction and degeneration of the neurons and glial cells of the retina.

As the disease progresses, severe nonproliferative diabetic retinopathy enters an advanced or proliferative (PDR) stage, where blood vessels proliferate/grow. The lack of oxygen in the retina causes fragile, new, blood vessels to grow along the retina and in the clear, gel-like vitreous humour that fills the inside of the eye. Without timely treatment, these new blood vessels can bleed, cloud vision, and destroy the retina. Fibrovascular proliferation can also cause tractional retinal detachment. The new blood vessels can also grow into the angle of the anterior chamber of the eye and cause neovascular glaucoma.

### Dry Eye Syndrome (DES)

“Dry eye syndrome (DES), also known as keratoconjunctivitis sicca (KCS), is the condition of having dry eyes. Other associated symptoms include irritation, redness, discharge, and easily fatigued eyes. Blurred vision may also occur. The symptoms can range from mild and occasional to severe and continuous. Scarring of the cornea may occur in untreated cases.

Dry eye occurs when either the eye does not produce enough tears or when the tears evaporate too quickly. This can result from contact lens use, meibomian gland dysfunction, pregnancy, Sjôgren syndrome, vitamin A deficiency, omega-3 fatty acid deficiency, LASIK surgery, and certain medications such as antihistamines, some blood pressure medication, hormone replacement therapy, and antidepressants. Chronic conjunctivitis such as from tobacco smoke exposure or infection may also lead to the condition.

### Glaucoma

Glaucoma is a group of eye diseases which result in damage to the optic nerve and cause vision loss. The most common type is open-angle (wide angle, chronic simple) glaucoma, in which the drainage angle for fluid within the eye remains open, with less common types including closed-angle (narrow angle, acute congestive) glaucoma and normal-tension glaucoma. Open-angle glaucoma develops slowly over time and there is no pain. Peripheral vision may begin to decrease, followed by central vision, resulting in blindness if not treated. Closed-angle glaucoma can present gradually or suddenly.

The underlying cause of open-angle glaucoma remains unclear. Several theories exist on its exact etiology. However, the major risk factor for most glaucomas and the focus of treatment is increased intraocular pressure. Intraocular pressure is a function of production of liquid aqueous humor by the ciliary processes of the eye, and its drainage through the trabecular meshwork. Aqueous humor flows from the ciliary processes into the posterior chamber, bounded posteriorly by the lens and the zonules of Zinn, and anteriorly by the iris. It then flows through the pupil of the iris into the anterior chamber, bounded posteriorly by the iris and anteriorly by the cornea. From here, the trabecular meshwork drains aqueous humor via the scleral venous sinus (Schlemm's canal) into scleral plexuses and general blood circulation.

### Uveitis and Noninfectious Uveitis (NIU)

Uveitis is the inflammation of the uvea, the pigmented layer that lies between the inner retina and the outer fibrous layer composed of the sclera and cornea. The uvea consists of the middle layer of pigmented vascular structures of the eye and includes the iris, ciliary body, and choroid. Uveitis is an ophthalmic emergency and requires a thorough examination by an ophthalmologist or optometrist and urgent treatment to control the inflammation. It is commonly associated with other ocular disorders, such as glaucoma, retinal detachment, optic nerve damage, cataracts, and, in some cases, a permanent loss of vision. The cause of non-infectious uveitis is unknown but there are some strong genetic factors that predispose disease onset including HLA-B27 and the PTPN22 genotype.

### Polypoidal Choroidal Vasculopathy

“Polypoidal choroidal vasculopathy” (PCV) is a disease of the choroidal vasculature. It is characterized by serosanguineous detachments of the pigmented epithelium and exudative changes that can commonly lead to subretinal fibrosis. PCV is characterized by abnormally shaped vessels in the choroid, but the precise causes of PCV remain unknown.

### Proliferative Vitreoretinopathy

“Proliferative vitreoretinopathy” (PVR) is a disease that develops as a complication of rhegmatogenous retinal detachment. PVR occurs in about 8-10% of patients undergoing primary retinal detachment surgery and prevents the successful surgical repair of rhegmatogenous retinal detachment.

PVR is graded as Grade A, B, or C by the Silicone Oil Study and as Grade A, B, C, or D by the Retina Society Terminology Committee. Grade A is characterized by the appearance of vitreous haze and RPE cells in the vitreous. Grade B is characterized by wrinkling of the edges of the retinal tear or the inner retinal surface. Grade C is characterized by the presence of retinal membranes.

### Retinal Vein Occlusion (RVO)

“Retinal vein occlusion” (RVO) affects the eye, specifically the retina. An occlusion occurs when one of the veins or arteries carrying blood to or from the retina becomes blocked or contains a blood clot. The blockage could occur in the main vein or main artery. Blockages could also occur in the branch of veins and arteries throughout the retina. A blockage in the vein or artery of the retina can cause blood or other fluids to build up and inhibit the retina's ability to filter light properly. When light is blocked or fluids are present, sudden loss of vision can occur. The severity of vision loss may be dependent upon where the blockage or clot occurred. Blockages in the main vein or artery are often more serious than blockages in the branch veins or arteries.

### Retinopathy of Prematurity (ROP)

“Retinopathy of prematurity” (ROP), also called retrolental fibroplasia (RLF) and Terry syndrome, is a disease of the eye affecting prematurely born babies generally having received neonatal intensive care, in which oxygen therapy is used due to the premature development of their lungs. It is thought to be caused by disorganized growth of retinal blood vessels which may result in scarring and retinal detachment. ROP can be mild and may resolve spontaneously, but it may lead to blindness in serious cases. Thus, all preterm babies are at risk for ROP, and very low birth-weight is an additional risk factor. Both oxygen toxicity and relative hypoxia can contribute to the development of ROP.

During development, blood vessels grow from the central part of the retina outwards. This process is completed a few weeks before the normal time of delivery. However, in premature babies it is incomplete. If blood vessels grow normally, ROP does not occur. If the vessels grow and branch abnormally the baby develops ROP. These abnormal blood vessels may grow up from the plane of the retina and may bleed inside the eye. When the blood and abnormal vessels are reabsorbed, it may give rise to multiple band like membranes which can pull up the retina, causing detachment of the retina and eventually blindness before 6 months.

### Retinal Ischemia

“Retinal ischemia” is the constellation of ocular signs and symptoms secondary to severe, chronic arterial hypoperfusion to the eye. Amaurosis fugax is a form of acute vision loss caused by reduced blood flow to the eye; it may be a warning sign of an impending stroke, as both stroke and retinal artery occlusion can be caused by thromboembolism due to atherosclerosis elsewhere in the body (such as coronary artery disease and especially carotid atherosclerosis). Consequently, those with transient blurring of vision are advised to urgently seek medical attention for a thorough evaluation of the carotid artery. Anterior segment ischemic syndrome is a similar ischemic condition of anterior segment usually seen in post-surgical cases. Retinal artery occlusion (such as central retinal artery occlusion or branch retinal artery occlusion) leads to rapid death of retinal cells, thereby resulting in severe loss of vision.

Example anti-CSF1 and anti-CSF1R antibodies are provided in Table 1A-B.

TABLE 1A
Example Anti-CSF1R Antibodies

| Antibody | Protein Sequences |
|---|---|
| Emactuzumab | Heavy chain (SEQ ID NO: 1) |
| (RG7155, or | QVQLVQSGAEVKKPGASVKVSCKASGYTFTSYDISWVRQAPGQGLEWMGVIWTDGGTNYA |
| RO5509554) | QKLQGRVTMTTDTSTSTAYMELRSLRSDDTAVYYCARDQRLYFDVWGQGTTVTVSSASTK |
| | GPSVFPLAPSSKSTSGGTAALGCLVKDYFPEPVTVSWNSGALTSGVHTFPAVLQSSGLYS |
| | LSSVVTVPSSSLGTQTYICNVNHKPSNTKVDKKVEPKSCDKTHTCPPCPAPELLGGPSVF |
| | LFPPKPKDTLMISRTPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQVYTLPPSRDELTKN |
| | QVSLTCLVKGFYPSDIAVEWESNGQPENNYKTTPPVLDSDGSFFLYSKLTVDKSRWQQGN |
| | VFSCSVMHEALHNHYTQKSLSLSPGK |
| | Light chain (SEQ ID NO: 2) |
| | DIQMTQSPSSLSASVGDRVTITCRASEDVNTYVSWYQQKPGKAPKLLIYAASNRYTGVPS |
| | RFSGSGSGTDFTLTISSLQPEDFATYYCQQSFSYPTFGQGTKLEIKRTVAAPSVFIFPPS |
| | DEQLKSGTASVVCLLNNFYPREAKVQWKVDNALQSGNSQESVTEQDSKDSTYSLSSTLTL |
| | SKADYEKHKVYACEVTHQGLSSPVTKSFNRGEC |
| Cabiralizumab | Heavy chain (SEQ ID NO: 3) |
| (FPA008) | QVQLVQSGAEVKKPGSSVKVSCKASGYTFTDNYMIWVRQAPGQGLEWMGDINPYNGGTTF |
| | NQKFKGRVTITADKSTSTAYMELSSLRSEDTAVYYCARESPYFSNLYVMDYWGQGTLVTV |
| | SSASTKGPSVFPLAPCSRSTSESTAALGCLVKDYFPEPVTVSWNSGALTSGVHTFPAVLQ |
| | SSGLYSLSSVVTVPSSSLGTKTYTCNVDHKPSNTKVDKRVESKYGPPCPPCPAPEFLGGP |
| | SVFLFPPKPKDTLMISRTPEVTCVVVDVSQEDPEVQFNWYVDGVEVHNAKTKPREEQFNS |
| | TYRVVSVLTVLHQDWLNGKEYKCKVSNKGLPSSIEKTISKAKGQPREPQVYTLPPSQEEM |
| | TKNQVSLTCLVKGFYPSDIAVEWESNGQPENNYKTTPPVLDSDGSFFLYSRLTVDKSRWQ |
| | EGNVFSCSVMHEALHNHYTQKSLSLSLGK |
| | Light chain (SEQ ID NO: 4) |
| | EIVLTQSPATLSLSPGERATLSCKASQSVDYDGDNYMNWYQQKPGQAPRLLIYAASNLES |
| | GIPARFSGSGSGTDFTLTISSLEPEDFAVYYCHLSNEDLSTFGGGTKVEIKRTVAAPSVF |
| | IFPPSDEQLKSGTASVVCLLNNFYPREAKVQWKVDNALQSGNSQESVTEQDSKDSTYSLS |
| | STLTLSKADYEKHKVYACEVTHQGLSSPVTKSFNRGEC |
| IMC-CS4 | Heavy chain (SEQ ID NO: 5) |
| (LY3022855) | QDQLVESGGGVVQPGRSLRLSCAASGFTFSSYGMHWVRQAPGEGLEWVAVIWYDGSNKYY |
| | ADSVKGRFTISRDNSKNTLYLQMNSLRAEDTAVYYCARGDYEVDYGMDVWGQGTTVTVAS |
| | ASTKGPSVFPLAPSSKSTSGGTAALGCLVKDYFPEPVTVSWNSGALTSGVHTFPAVLQSS |
| | GLYSLSSVVTVPSSSLGTQTYICNVNHKPSNTKVDKRVEPKSCDKTHTCPPCPAPELLGG |
| | PSVFLFPPKPKDTLMISRTPEVTCVVVDVSHEDPEVKFNWYVDGVEVHNAKTKPREEQYN |
| | STYRVVSVLTVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSKLTVDKSRW |
| | QQGNVFSCSVMHEALHNHYTQKSLSLSPGK |
| | Light chain (SEQ ID NO: 6) |
| | AIQLTQSPSSLSASVGDRVTITCRASQGISNALAWYQQKPGKAPKLLIYDASSLESGVPS |
| | RFSGSGSGTDFTLTISSLQPEDFATYYCQQFNSYPWTFGQGTKVEIKRTVAAPSVFIFPP |
| | SDEQLKSGTASVVCLLNNFYPREAKVQWKVDNALQSGNSQESVTEQDSKDSTYSLSSTLT |
| | LSKADYEKHKVYACEVTHQGLSSPVTKSFNRGEC |
| AM001 | Heavy chain (SEQ ID NO: 7) |
| | QVQLVQSGAEVKKPGASVKVSCKASGYTFTSYGISWVRQAPGQGLEWMGWISAYNGNTNY |
| | AQKLQGRVTMTTDTSTSTAYMELRSLRSDDTAVYYCARESWFGEVFFDYWGQGTLVTVSS |
| | ASTKGPSVFPLAPCSRSTSESTAALGCLVKDYFPEPVTVSWNSGALTSGVHTFPAVLQSS |
| | GLYSLSSVVTVPSSNFGTQTYTCNVDHKPSNTKVDKTVERKCCVECPPCPAPPVAGPSVF |
| | LFPPKPKDTLMISRTPEVTCVVVDVSHEDPEVQFNWYVDGVEVHNAKTKPREEQFNSTFR |
| | VVSVLTVVHQDWLNGKEYKCKVSNKGLPAPIEKTISKTKGQPREPQVYTLPPSREEMTKN |
| | QVSLTCLVKGFYPSDIAVEWESNGQPENNYKTTPPMLDSDGSFFLYSKLTVDKSRWQQGN |
| | VFSCSVMHEALHNHYTQKSLSLSPGK |
| | Light chain (SEQ ID NO: 8) |
| | DIVMTQSPDSLAVSLGERATINCKSSQSVLDSSDNKNYLAWYQQKPGQPPKLLIYWASNR |
| | ESGVPDRFSGSGSGTDFTLTISSLQAEDVAVYYCQQYYSDPFTFGPGTKVDIKRTVAAPS |
| | VFIFPPSDEQLKSGTASVVCLLNNFYPREAKVQWKVDNALQSGNSQESVTEQDSKDSTYS |
| | LSSTLTLSKADYEKHKVYACEVTHQGLSSPVTKSFNRGEC |
| Axatilimab | Heavy chain (SEQ ID NO: 9) |
| (SNDX-6352) | EVTLKESGPALVKPTQTLTLTCTFSGFSLTTYGMGVGWIRQPPGKALEWLANIWWDDDKY |
| | YNPSLKNRLTISKDTSKNQVVLTMTNMDPVDTATYYCARIGPIKYPTAPYRYFDFWGQGT |
| | MVTVSSASTKGPSVFPLAPCSRSTSESTAALGCLVKDYFPEPVTVSWNSGALTSGVHTFP |
| | AVLQSSGLYSLSSVVTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYVDGVEVHNAKTKPREE |
| | QFNSTYRVVSVLTVLHQDWLNGKEYKCKVSNKGLPSSIEKTISKAKGQPREPQVYTLPPS |
| | QEEMTKNQVSLTCLVKGFYPSDIAVEWESNGQPENNYKTTPPVLDSDGSFFLYSRLTVDK |
| | SRWQEGNVFSCSVMHEALHNHYTQKSLSLSLGK |
| | Light chain (SEQ ID NO: 10) |
| | DIQMTQSPSSLSASVGDRVTITCLASEDIYDNLAWYQQKPGKAPKLLIYYASSLQDGVPS |
| | RFSGSGSGTDYTLTISSLQPEDFATYYCLQDSEYPWTFGGGTKVEIKRTVAAPSVFIFPP |
| | SDEQLKSGTASVVCLLNNFYPREAKVQWKVDNALQSGNSQESVTEQDSKDSTYSLSSTLT |
| | LSKADYEKHKVYACEVTHQGLSSPVTKSFNRGEC |

TABLE 1B
Example Anti-CSF1 Antibodies

| Antibody | Protein Sequences |
|---|---|
| Lacnotuzumab | Heavy chain (SEQ ID NO: 11) |
| (MCS110) | QVQLQESGPGLVKPSQTLSLTCTVSDYSITSDYAWNWIRQFPGKGLEWMGYISYSGSTSY |
| | NPSLKSRITISRDTSKNQFSLQLNSVTAADTAVYYCASFDYAHAMDYWGQGTTVTVSSAS |
| | TKGPSVFPLAPSSKSTSGGTAALGCLVKDYFPEPVTVSWNSGALTSGVHTFPAVLQSSGL |
| | YSLSSVVTVPSSSLGTQTYICNVNHKPSNTKVDKRVEPKSCDKTHTCPPCPAPELLGGPS |
| | VFLFPPKPKDTLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQVYTLPPSREEMT |
| | KNQVSLTCLVKGFYPSDIAVEWESNGQPENNYKTTPPVLDSDGSFFLYSKLTVDKSRWQQ |
| | GNVFSCSVMHEALHNHYTQKSLSLSPGK |
| | Light chain (SEQ ID NO: 12) |
| | DIVLTQSPAFLSVTPGEKVTFTCQASQSIGTSIHWYQQKTDQAPKLLIKYASESISGIPS |
| | RFSGSGSGTDFTLTISSVEAEDAADYYCQQINSWPTTFGGGTKLEIKRTVAAPSVFIFPP |
| | SDEQLKSGTASVVCLLNNFYPREAKVQWKVDNALQSGNSQESVTEQDSKDSTYSLSSTLT |
| | LSKADYEKHKVYACEVTHQGLSSPVTKSFNRGEC |
| MCS110 var | Heavy chain (SEQ ID NO: 13) |
| | DVQLQESGPGLVKPSQSLSLTCTVTDYSITSDYAWNWIRQFPGNKLEWMGYISYSGSTSY |
| | NPSLKSRISITRDTSKNQFFLQLNSVTTEDTATYYCASFDYAHAMDYWGQGTSVTVSSAK |
| | TTAPSVYPLAPVCGDTTGSSVTLGCLVKGYFPEPVTLTWNSGSLSSGVHTFPAVLQSDLY |
| | TLSSSVTVTSSTWPSQSITCNVAHPASSTKVDKKIEPRGPTIKPCPPCKCPAPNLLGGPS |
| | VFIFPPKIKDVLMISLSPIVTCVVVDVSEDDPDVQISWFVNNVEVHTAQTQTHREDYNST |
| | LRVVSALPIQHQDWMSGKEFKCKVNNKDLPAPIERTISKPKGSVRAPQVYVLPPPEEEMT |
| | KKQVTLTCMVTDFMPEDIYVEWTNNGKTELNYKNTEPVLDSDGSYFMYSKLRVEKKNWVE |
| | RNSYSCSVVHEGLHNHHTTKSFSRTPG |
| | Light chain (SEQ ID NO: 14) |
| | DILLTQSPAILSVSPGERVSFSCRASQSIGTSIHWYQQRTNGSPRLLIKYASESISGIPS |
| | RFSGSGSGTDFTLSINSVESEDIADYYCQQINSWPTTFGGGTKLEIKRADAAPTVSIFPP |
| | SSEQLTSGGASVVCFLNNFYPKDINVKWKIDGSERQNGVLNSWTDQDSKDSTYSMSSTLT |
| | LTKDEYERHNSYTCEATHKTSTSPIVKSFNRNEC |
| PD-0360324 | Heavy chain (SEQ ID NO: 15) |
| | EVQLVESGGGLVQPGGSLRLSCAASGFTFSSFSMTWVRQAPGKGLEWVSYISSRSSTISY |
| | ADSVKGRFTISRDNAKNSLYLQMNSLRDEDTAVYYCARDPLLAGATFFDYWGQGTLVTVS |
| | SASTKGPSVFPLAPCSRSTSESTAALGCLVKDYFPEPVTVSWNSGALTSGVHTFPAVLQS |
| | SGLYSLSSVVTVPSSNFGTQTYTCNVDHKPSNTKVDKTVERKCCVECPPCPAPPVAGPSV |
| | FLFPPKPKDTLMISRTPEVTCVVVDVSHEDPEVQFNWYVDGVEVHNAKTKPREEQFNSTF |
| | RVVSVLTVVHQDWLNGKEYKCKVSNKGLPAPIEKTISKTKGQPREPQVYTLPPSREEMTK |
| | NQVSLTCLVKGFYPSDIAVEWESNGQPENNYKTTPPMLDSDGSFFLYSKLTVDKSRWQQG |
| | NVFSCSVMHEALHNHYTQKSLSLSPGK |
| | Light chain (SEQ ID NO: 16) |
| | EFVLTQSPGTLSLSPGERATLSCRASQSVSSSYLAWYQQKPGQAPRLLIYGASSRATGIP |
| | DRFSGSGSGTDFTLTISRLEPEDFAVYYCQQYGSSPLTFGGGTKVEIKRTVAAPSVFIFP |
| | PSDEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC |

Emactuzumab (also known as RG7155 and R05509554) is a clinical stage humanized IgG1 CSF1R targeted antibody designed to target and deplete macrophages in the tumor tissue. It has shown a favorable safety profile in patients and encouraging efficacy for TGCT. Emactuzumab is under investigation in clinical trial NCT01494688—“A Study of R05509554 as Monotherapy and in Combination with Paclitaxel in Participants With Advanced Solid Tumors.”

Cabiralizumab (also known as FPA008) is under investigation in clinical trial NCT03502330—“APX005M With Nivolumab and Cabiralizumab in Advanced Melanoma, Non-small Cell Lung Cancer or Renal Cell Carcinoma.” Cabiralizumab is a humanized IgG4 anti-CSF1R monoclonal antibody with a single amino acid substitution in the hinge region to prevent hemi-dimer exchange.

IMC-CS4 (also known as LY3022855) is a human IgG1 antibody (mAb) targeting CSF1R. IMC-CS4 is under investigation in clinical trial NCT01346358—“A Study of IMC-CS4 in Subjects With Advanced Solid Tumors.”

Axatilimab (also known as SNDX-6352) is a humanized, full-length IgG4 antibody with high affinity to CSF-1R. Axatilimab affects the migration, proliferation, differentiation, and survival of monocytes and macrophages by binding to CSF-1R and blocking its activation by its two known ligands, CSF-1 and IL-34. Axatilimab is currently being evaluated in a Phase 1/2 clinical trial in patients with cGVHD.

Lacnotuzumab (also known as MCS110) is a high-affinity human engineered IgG1 anti-CSF1 antibody that blocks the ability of CSF1R to drive proliferation in responsive cells. Lacnotuzumab is under investigation in clinical trial NCT01643850—“MCS110 in Patients With Pigmented Villonodular Synovitis (PVNS).”

PD-0360324 is a fully human immunoglobulin G2 monoclonal antibody against CSF1 investigated for treating cutaneous lupus erythematosus (CLE). It is also being tested for its combination with Cyclophosphamide in treating patients with recurrent high-grade epithelial ovarian, primary peritoneal, or fallopian tube cancer.

AM001 is a fully human IgG2 anti-CSF1R antibody. AM001 has a heavy chain variable region of SEQ ID NO:7 and a light chain variable region of SEQ ID NO:8 which were prepared and tested in PCT application WO 2009/026303. The epitopes are mainly located at the N-terminus Ig-like loop 1 and Ig-like loop 2 of human CSF1R, and requires the presence of both the loop 1 and loop 2 regions.

In some embodiments, an anti-VEGF inhibitor is also administered. In some embodiments, the anti-VEGF inhibitor is a small molecule. In some embodiments, the anti-VEGF inhibitor is an antibody. In some embodiments, the inhibitor inhibits VEGF-A. In some embodiments, the inhibitor inhibits placenta growth factor (PGF). In some embodiments, the inhibitor inhibits VEGF-B. In some embodiments, the inhibitor inhibits VEGF-C. In some embodiments, the inhibitor inhibits VEGF-D.

Non-limiting examples of VEGF inhibitors include sorafenib (Nexavar, Bayer), sunitinib (Sutent, Pfizer), bevacizumab (Avastin, Genentech), pazopanib (Votrient, Novartis), axitinib (Inlyta, Pfizer), cabozantinib (Cometriq, Exelixis), ranibizumab (Lucentis), and lenvatinib (Lenvima, Eisai).

The CSF1R or CSF1 antibodies or the anti-VEGF inhibitors can be administered to the patient by methods known in the art. The administration, for instance, may be via intradermal, intramuscular, intraperitoneal, intravenous, subcutaneous, intranasal, epidural, and oral routes. A preferred route of administration is intravitreal injection.

In some embodiments, the CSF1R or CSF1 antibody is combined with an anti-VEGF antibody to form a bispecific antibody. A bispecific antibody can adopt any format known in the art.

In one embodiment, the bispecific antibody includes a single chain fragment (scFv) specific to CSF1R or CSF1 and a second scFv specific to VEGF. A scFv can be prepared by fusing a heavy chain variable region (VH) of an antibody with a light chain (VL) of the antibody through a peptide linker. Therefore, any antibody disclosed herein can be readily adapted to a scFv for inclusion in a bispecific antibody.

In one embodiment, the VH is at the N-terminal side of the VL; in another embodiment, the VH is at the C-terminal side of the VL.

In some embodiments, the bispecific antibody is monovalent for each target. An example is that one copy of each of the scFv is fused to one chain of a Fc fragment. In another example, one of the monovalent unit includes a scFv and the other includes a conventional Fab fragment, which constitutes an asymmetrical antibody format.

In some embodiments, the bispecific antibody is bivalent for at least one of the targets. For instance, two scFv have the same specificity can be concatenated forming a bivalent unit, while the other specificity is monovalent. In some embodiment, two Fab fragments can be concatenated forming a bivalent unit, while the other specificity is monovalent.

In some embodiments, the bispecific antibody is bivalent for both of the targets. For instance, two scFv specific to CSF1R or CSF1 can be fused to the C-terminal end of a conventional anti-VEGF antibody. Likewise, two scFv specific to VEGF can be fused to the C-terminal end of a conventional anti-CSF1R or CSF1 antibody.

Non-limiting examples of VEGF antibodies include (Avastin, Genentech) and ranibizumab (Lucentis).

New antibodies for CSF1R, CSF1 or VEGF can also be developed, such as single domain antibodies (nanobodies). Nanobodies are single chain molecule and thus can be readily integrated into a bispecific format, like a scFv.

In some embodiments, the VEGF inhibitor is a VEGF-trap. VEGF-R like Aflibercept/Eylea

The method of claim 9, wherein the VEGF inhibitor is a VEGF-trap, which binds to VEGF like a receptor but instead inhibits the activity of VEGF. An example is Aflibercept (Eylea®). In some embodiments, the antibody and the VEGF-trap are provided as a bifunctional molecule, such as a fusion protein, with the VEGF-trap fused to one of the Fc chains.

It is contemplated that, through intravitreal injection, the anti-CSF1R or anti-CSF1 antibodies can stay effective in the eye for an extended period of time. Accordingly, the administration can be only once every one, two, three, four, five, or six months.

As a general proposition, the dosage administered to a patient of the antibody or antigen-binding polypeptides of the present disclosure is typically 0.1 mg/kg to 100 mg/kg of the patient's body weight, between 0.1 mg/kg and 20 mg/kg of the patient's body weight, or 1 mg/kg to 10 mg/kg of the patient's body weight. In some embodiments, lower dosages of human antibodies and less frequent administration is often possible.

The present disclosure, in one embodiment, provides aqueous formulations of an anti-CSF1 or anti-CSF1R antibody or fragment suitable for the treatment methods. In some embodiments, the aqueous formulations have relatively high concentration of the antibody or fragment, e.g., at a concentration that is at least about 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, 200 mg/mL, 210 mg/mL, 220 mg/mL, 230 mg/mL, 240 mg/mL, or even 250 mg/mL. In some embodiments, the concentration is not higher than about 500 mg/mL, 400 mg/mL, or 300 mg/mL.

In some embodiments, the formulation includes one or more tonicity agents. The term “tonicity agent” as used herein denotes pharmaceutically acceptable agents used to modulate the tonicity of the formulation. Isotonicity generally relates to the osmotic pressure relative to a solution, usually relative to that of human blood serum. A formulation can be hypotonic, isotonic or hypertonic. In one aspect, the formulation is isotonic. An isotonic formulation is liquid or liquid reconstituted from a solid form, or suspension that solubilize up on diluation, e.g. from a lyophilized form and denotes a solution having the same tonicity as some other solution with which it is compared, such as physiologic salt solution and the blood serum. Suitable isotonicity agents include but are not limited to sodium chloride, potassium chloride, glycerin and any component from the group of amino acids, sugars, as defined herein as well as combinations thereof.

In some embodiments, the formulation includes one or more surfactants. As used herein, the term “surfactant” refers to a pharmaceutically acceptable organic substance having amphipathic structures; namely, it is composed of groups of opposing solubility tendencies, typically an oil-soluble hydrocarbon chain and a water-soluble ionic group. Surfactants can be classified, depending on the charge of the surface-active moiety, into anionic, cationic, and nonionic surfactants. Surfactants are often used as wetting, emulsifying, solubilizing, and dispersing agents for various pharmaceutical formulations and preparations of biological materials. In some embodiments of the pharmaceutical formulations described herein, the amount of surfactant is described as a percentage expressed in weight/volume percent (w/v %). Suitable pharmaceutically acceptable surfactants include but are not limited to the group of polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), or sodium dodecyl sulphate (SDS). Polyoxyethylenesorbitan-fatty acid esters include polysorbate 20, (sold under the trademark Tween 20™) and polysorbate 80 (sold under the trademark Tween 80™). Polyethylene-polypropylene copolymers include those sold under the names Pluronic® F68 or Poloxamer 188™. Polyoxyethylene alkyl ethers include those sold under the trademark Brij™. Alkylphenolpolyoxyethylene ethers include those sold under the tradename Triton-X.

In some embodiments, the formulation includes one or more lyoprotectants. A “lyoprotectant” refers to a pharmaceutically acceptable substance that stabilizes a protein during lyophilization (the process of rapid freezing and drying in a high vacuum). Examples of lyoprotectants include, without limitation, sucrose, trehalose or mannitol.

In some embodiments, the formulation further includes one or more antioxidants. An “antioxidant” refers to a molecule capable of slowing or preventing the oxidation of other molecules. Oxidation is a chemical reaction that transfers electrons from a substance to an oxidizing agent. Oxidation reactions can produce free radicals, which start chain reactions that destabilize the protein therapeutics and ultimately affect the product activity. Antioxidants terminate these chain reactions by removing free radical intermediates and inhibit other oxidation reactions by being oxidized themselves. As a result, antioxidants are often reducing agents, chelating agent and oxygen scavengers such as citrate, EDTA, DPTA, thiols, ascorbic acid or polyphenols. Non-limiting examples of antioxidants include ascorbic acid (AA, E300), thiosulfate, methionine, tocopherols (E306), propyl gallate (PG, E310), tertiary butylhydroquinone (TBHQ), butylated hydroxyanisole (BHA, E320) and butylated hydroxytoluene (BHT, E321).

In some embodiments, the formulation further includes one or more preservatives. A “preservative” is a natural or synthetic chemical that is added to products such as foods, pharmaceuticals, paints, biological samples, wood, etc. to prevent deformulation by microbial growth or by undesirable chemical changes. Preservative additives can be used alone or in conjunction with other methods of preservation. Preservatives may be antimicrobial preservatives, which inhibit the growth of bacteria and fungi, or antioxidants such as oxygen absorbers, which inhibit the oxidation of constituents. Common antimicrobial preservatives include, benzalkonium chloride, benzoic acid, cholorohexidine, glycerin, phenol, potassium sorbate, thimerosal, sulfites (sulfur dioxide, sodium bisulfite, potassium hydrogen sulfite, etc.) and disodium EDTA. Other preservatives include those commonly used in patenteral proteins such as benzyl alcohol, phenol, m-cresol, chlorobutanol or methylparaben.

In some embodiments, the formulation further includes one or more bulking agents. As used herein, the term “bulking agent” refers to an ingredient that provides bulk to a lyophilized formulation. Examples of bulking agents include, without limitation, mannitol, trehalose, lactose, sucrose, polyvinyl pyrrolidone, sucrose, glucose, glycine, cyclodextrins, dextran, solid PEGs and derivatives and mixtures thereof. In one embodiment, a formulation of the present disclosure optionally includes a bulking agent.

In some embodiment the formulation further includes buffering system such as citrate, acetate, borate, phosphate or combination of. In some embodiment the formulation further includes tertiary butanol to enhance property and stability of lyophilized material.

In some embodiment the formulation further includes viscosity lowering agent such as lysin, arginine, NaCl, glutamine, glycine or combinations thereof.

In some embodiments, the formulation can include a controlled-release or stabilization polymer that may be selected from hyaluronic acid (HA), alginate, hydroxy methylcellulose (HPMC), hydroxy propylcellulose (HPC), sodium carboxymethyl cellulose (NaCMC); or povidones. Biodegradable matrices may comprise excipients such as, poly(D,L-lactic acid) (PLA), poly(D,L-lactic-co-glycolic acid) (PLGA), or a block copolymer comprising hydrophilic poly(ethylene glycol) (PEG) and one or more polymers selected from poly(lactic acid-co-glycolic acid) (PLGA), poly(ε-caprolactone) (PCL), and poly(ε-caprolactone-co-glycolic acid) (PCGA), such as poly(ε-caprolactone-co-glycolic acid)-poly(ethylene glycol)-poly(ε-caprolactone-co-glycolic acid) (PCGA-PEG-PCGA) and poly(lactic acid-co-glycolic acid)-poly(ethylene glycol)-poly(lactic acid-co-glycolic acid) (PLGA-PEG-PLGA) or a pharmaceutically acceptable salt thereof, or a combination thereof. In some embodiments, the formulation can include HSA (human serum albumin) or BSA (bovine serum albumin).

In some embodiments, the formulation (e.g., suspension) also includes suspending agent. The term “suspending agent” as used herein refers to a pharmaceutical acceptable excipient that promotes particle suspension or dispersion and reduces sedimentation. Suspending agents also act as thickening agents. They increase in viscosity of the solution, which is helpful to prevent sedimentation of the suspended particles. A suspension has well developed thixotropy. At rest the solution is sufficient viscous to prevent sedimentation and thus aggregation or caking of the particles. When agitation is applied the viscosity is reduced and provide good flow characteristic.

Non-limiting examples of types of suspending agents include polysaccharides, inorganic salts, and polymers. Specific examples of suspending agents include, without limitation, alginates, methylcellulose, hydroxyethylcellulose, carboxymethylcellulose, carboxymethylcellulose sodium, microcrystalline cellulose, acacia, tragacanth, xanthan gum, bentonite, carbomer, carageenan, powdered cellulose, gelatin, polyethylene glycol, povidone, dextrin, medium-chain triglycerides, sucrose, hydroxypropyl methyl cellulose, chistosan, polyoxyethylene, polyoxy-propylene ethers and combinations thereof.

In some embodiments, the suspending agent is selected from polyethylene glycol (e.g., polyethylene glycol 4000), carboxymethylcellulose sodium, methylcellulose, povidone, and combinations thereof. In one embodiment, the suspending agent is polyethylene glycol 4000. In another embodiment, the suspending agent is carboxymethylcellulose sodium.

The concentration of the suspending agent can generally be from about 0.1 mg/mL to about 200 mg/mL, or from about 0.5 mg/mL to about 100 mg/mL, from about 1 mg/mL to about 75 mg/mL, from about 5 mg/mL to about 60 mg/mL, from about 5 mg/mL to about 20 mg/mL, or from about 40 mg/mL to about 60 mg/mL. In some embodiments, concentration of the suspending agent is from about 0.1% (w/w) to about 7.5% (w/w), or from about 0.1% (w/w) to about 6% (w/w), from about 0.2% (w/w) to about 6% (w/w), from about 0.5% (w/w) to about 6% (w/w), from about 1% (w/w) to about 6% (w/w).

For polyethylene glycol 4000, the concentration can be from about 10 mg/mL to about 100 mg/mL, from about 25 mg/mL to about 75 mg/mL, from about 40 mg/mL to about 70 mg/mL, or from about 50 mg/mL to about 60 mg/mL. For carboxymethylcellulose sodium, the concentration can be from about 1 mg/mL to about 50 mg/mL, from about 2 mg/mL to about 30 mg/mL, from about 5 mg/mL to about 20 mg/mL, or from about 7 mg/mL to about 15 mg/mL. For methylcellulose, the concentration can be from about 0.1 mg/mL to about 10 mg/mL, from about 0.2 mg/mL to about 5 mg/mL, from about 0.5 mg/mL to about 2 mg/mL, or from about 0.75 mg/mL to about 1.25 mg/mL.

Also provided, in some embodiments, is a lyophilized composition that can be prepared by freeze-drying the aqueous solution as disclosed herein. In some embodiments, also provided is a solution that can be prepared by dissolving the lyophilized composition in a solvent such as water.

### EXAMPLES

### Example 1. Treatment of wetAMD in LCNV Model

This example tested the efficacy of M279 in treating wet, or exudative, form of age-related macular degeneration (wetAMD) in a LCNV (laser-induced choroidal neovascularization) mouse model. M279 is the mouse surrogate for the anti-human CSF1R antibody AM001.

Laser-induced rupture of Bruch's membrane was used to generate choroidal neovascularization (CNV) in the eyes of 6-week-old male C57BL/6 mice (Charles River; Wilmington, MA). Mice were anesthetized with an intraperitoneal injection of ketamine/xylazine, and a 1% tropicamide solution was used to dilate the eyes. Using a hand-held cover slip as contact lens and GenTeal lubricating eye gel as a medium contacting the cover slip to the surface of the cornea, a GYC-500 green laser photocoagulator coupled to a SL-1800 slit-lamp was used to create four lesions equidistant from the optic nerve head in the retinal mid-periphery. At one day post-laser (FIG. 1), mice were treated with intravitreal injections of vehicle control, anti-VEGF antibody, and M279 (low, medium, and high doses).

Fluorescein Angiography. At 6 days post-laser (FIG. 1), vascular permeability was assessed via fluorescein angiography (FA). Mice were anesthetized with an intraperitoneal injection of ketamine/xylazine. Sodium fluorescein was injected intraperitoneally at 10 μl/g body weight. Fluorescent fundus images were captured with a imaging system at 2 and 4 minutes after injection. Image software was used to quantify the fluorescence intensity of the images using ‘integrated density’. The difference in integrated density between 2 and 4 minutes was recorded as a readout of vascular leakage.

CNVArea Quantification. At 7 days post-laser (FIG. 1), the extent of CNV at the Bruch's membrane rupture sites was measured using computer-assisted image analysis of choroidal flat-mounts stained using FITC-conjugated isolectin B4. The average area for all lesions in both eyes of each mouse was reported as a single data point.

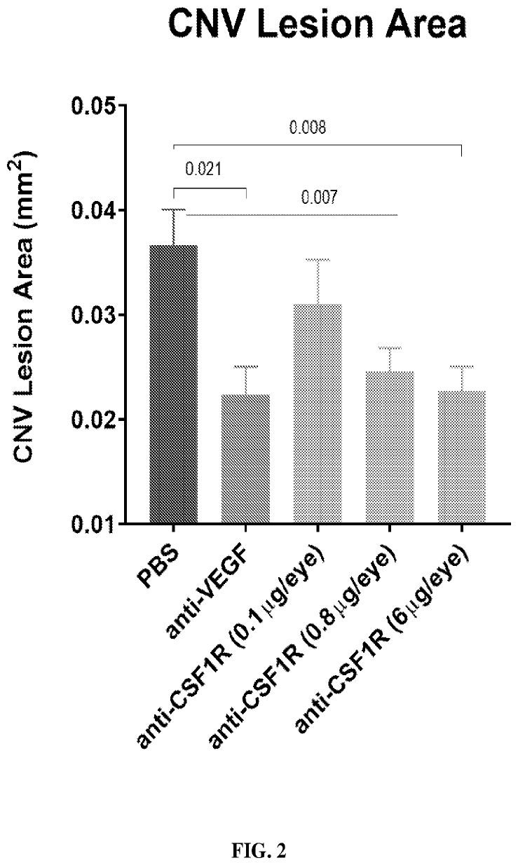

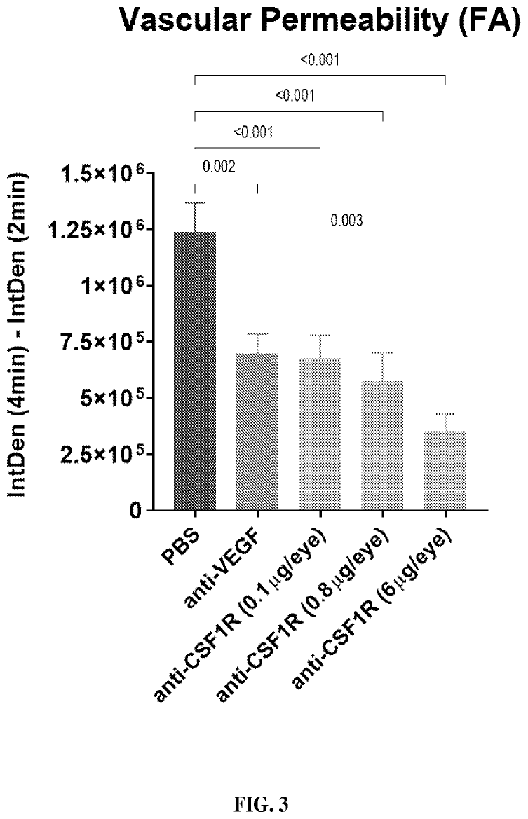

The results are presented in FIGS. 2 and 3. The reference anti-VEGF antibody was able to reduce the CNV lesion area significantly. Likewise, M279 dose-dependently significantly attenuated choroidal neovascularization (FIG. 2). In terms of reduction of vascular permeability, the efficacy of M279 was even more pronounced than the reference anti-VEGF antibody even at low doses and statistically so at the 6 μg/eye dose (FIG. 3).

### Example 2. Treatment of PDR in OIR Model

This example proposes to test the efficacy of M279 in treating proliferative diabetic retinopathy (PDR) in an OIR (oxygen-induced retinopathy) mouse model.

The ORI model is a widely used system to examine ocular neovascularization, a condition which resembles proliferative diabetic retinopathy in humans. Central area of established vasculature is obliterated by hyperoxic insult, creating an ischemic area. After mice are returned to room air, revascularization occurs and the normal vasculature is recovered, although some vessels (i.e., neovascular tufts [NVTs]) proliferate abnormally toward the vitreous body. M279 is a mouse surrogate for the anti-human CSF1R antibody AM001.

C57/BL6 mouse pups will be born and raised with dams in room air (RA; 20.9%) for 7 days (P0-P7), and then moved to a 75% oxygen atmosphere for 5 days (P8-P12) before removal to RA. At P12, all mice will receive intravitreal injections of vehicle control, an anti-VEGF antibody, M279 (low and high doses), or M279+anti-VEGF antibody. The same treatment will be administered to both eyes of each animal. Mice are anesthetized with isoflurane and treated with topical proparacaine before injection. Intravitreal injections of 1.0 μL volume are given. All mice will be sacrificed on P17, five days after removal from the oxygen exposure chamber. Both normal, intra-retinal vascular growth and pathological, pre-retinal neovascularization will be assessed in isolectin-B4-stained retinas. Areas of normal and abnormal vascular growth will be measured via image analysis.

TABLE 2A
M279 dose-response testing groups

| Group # | Treatment | Dose Level | |
|---|---|---|---|
| 1 | Vehicle | PBS | |
| 2 | anti-VEGF reference | 0.1 | μg/eye |
| 3 | M279 low | 0.1 | μg/eye |
| 4 | M279 medium | 0.8 | μg/eye |
| 5 | M279 high | 6 | μg/eye |

Combination therapy evaluation of anti-VEGF and anti-CSF1R may be performed in the LCNV and/or OIR models. The test groups would be as described below:

TABLE 2B
Combination testing groups

| Group # | Treatment | Dose Level |
|---|---|---|
| 1 | Vehicle | PBS |
| 2 | anti-VEGF mAb | 0.1 μg/eye |
| 3-4 | M279 | 0.1 & 6 μg/eye |
| 5-6 | M279 + anti-VEGF mAb | 0.1 or 6 μg M279 + |
| | | 0.1 μg anti-VEGF mAb/eye |

### Example 3. Treatment of Fibrosis in a Wet AMD Model

This example conducts an experiment similar to Example 1 except no vascular permeability analysis and CNV and fibrosis endpoints are measured 14 days following IVT therapeutic administration (15 days post-laser). Fibrosis endpoints are analyses of fibronectin, alpha-SMA, and collagen staining in the CNV areas. 4 test groups: vehicle, anti-VEGF mAb, and low and high M279 doses.

### Example 4. Treatment of DME in STZ-Treated Mice Model

This example proposes to test the efficacy of M279 for treating diabetic macular edema (DME) in mice treated with streptozotocin (STZ), a generally-recognized preclinical model for DME. M279 is a mouse surrogate for the anti-human CSF1R antibody AM00L.

Male C57BL/6J mice, 6 to 8 weeks old, receive daily IP injections of 50 mg STZ/kg body weight for 5 consecutive days. Mice with fasting blood glucose levels 300 mg/dL 7 days after the first STZ injection are deemed diabetic. Mice will be treated with intravitreal injections of vehicle control, anti-VEGF antibody, M279 (low and high doses), or M279+anti-VEGF antibody once a week beginning at 8 weeks post-diabetes induction. The same treatment will be administered to both eyes of each animal. Retinal vasopermeability, assessed via Evans blue dye (EBD) extravasation, and retinal leukostasis, assessed via concanavalin-A perfusion, are evaluated at 10 weeks post-diabetes induction.

Although this disclosure has been described with reference to the disclosed embodiments, those skilled in the art will readily appreciate that the specific examples and studies detailed above are only illustrative of this disclosure. It should be understood that various modifications can be made without departing from the spirit of this disclosure. Accordingly, this disclosure is limited only by the following claims.

1. A method for treating an ophthalmic disease in a human patient in need thereof, comprising administering to the patient an effective amount of a CSF1R inhibitor.

2. The method of claim 1, wherein the ophthalmic disease is characterized with vascular hyperpermeability.

3. The method of claim 1, wherein the ophthalmic disease is characterized with neovascularization.

4. The method of claim 1, wherein the ophthalmic disease is characterized with fibrosis, which is optionally subretinal macular fibrosis.

5. The method of claim 1, wherein the ophthalmic disease is selected from the group consisting of age-related macular degeneration (AMD), anterior segment neovascularization, central retinal vein occlusion (CRVO), choroidal neovascularization (CNV), corneal neovascularization, diabetic macular edema (DME), diabetic retinopathy, dry eye syndrome (DES), glaucoma, noninfectious uveitis (NIU), polypoidal choroidal vasculopathy, posterior segment neovascularization, proliferative diabetic retinopathy (PDR), proliferative vitreoretinopathy, retinal edema, retinal vein occlusion (RVO), retinopathy of prematurity (ROP), sequela associated with retinal ischemia, and uveitis.

6. The method of claim 4, wherein the ophthalmic disease is AMD, which is optionally exudative AMD (wetAMD or neovascular AMD).

7. The method of claim 4, wherein the ophthalmic disease is PDR.

8. The method of claim 4, wherein the ophthalmic disease is DME.

9. The method of claim 1, further comprising administering to the patient a VEGF inhibitor, which is optionally formulated together with the CSF1R inhibitor, or administered separately from the CSF1R inhibitor.

10. The method of claim 9, wherein the VEGF inhibitor is an anti-VEGF antibody.

11. The method of claim 1, wherein the CSF1R inhibitor is an anti-CSF1R antibody.

12. The method of claim 11, wherein the anti-CSF1R antibody and the anti-VEGF antibody are provided as a bispecific antibody having specificity to both CSF1R and VEGF.

13. The method of claim 9, wherein the VEGF inhibitor is a VEGF-trap.

14. The method of claim 13, wherein the VEGF-trap and the anti-CSF1R antibody are provided as bifunctional molecule.

15. The method of claim 11, wherein the anti-CSF1R antibody is selected from the group consisting of emactuzumab, cabiralizumab, IMC-CS4, AM001, axatilimab, and antigen-binding fragments thereof.

16. The method of claim 11, wherein the anti-CSF1R antibody is AM001 or an antigen-binding fragment thereof.

17. The method of claim 1, wherein the administration results in reduction or prevention of ocular fibrosis.

18. The method of claim 1, wherein the administration is intravitreal injection.

19. The method of claim 18, wherein the administration is once every one, two, three, four, five, or six months.
